(19) United States
(12) Patent Application Publication
(10) Pub. No.: US 2025/0303133 A1
(43) Pub. Date: Oct. 2, 2025

(54) DRUG DELIVERY BEYOND THE BLOOD-BRAIN BARRIER USING SHOCK WAVES

(71) Applicant: Shockwave Medical, Inc., Santa Clara, CA (US)

(72) Inventors: Kemal SARIIBRAHIMOGLU, Santa Clara, CA (US); Thomas Charles HASENBERG, Campbell, CA (US); Show-Mean Steve WU, Santa Clara, CA (US)

(73) Assignee: Shockwave Medical, Inc., Santa Clara, CA (US)

(21) Appl. No.: 18/620,248

(22) Filed: Mar. 28, 2024

(51) Int. Cl. A61M 37/00; A61M 3/02; A61M 31/00

(52) U.S. Cl. CPC A61M 37/0092; A61M 3/0275; A61M 31/005; A61M 2037/0007; A61M 2210/0681

(57) ABSTRACT

Described herein are shock wave catheters and methods of use thereof for delivering an active agent of a drug to the CNS via a body lumen or cavity, such as sinonasal cavity, which can bypass the blood-brain barrier (BBB). The catheters described herein can be advanced through an intranasal passage to the nasal cavity such that at least a portion of the enclosure is disposed in the nasal cavity. The enclosure can be coated with a drug coating. The method can include filling the enclosure with a conductive fluid. At least one shock wave can be generated at a shock wave emitter of the catheter disposed within the enclosure. The at least one shock wave can cause a therapeutically effective amount of the active agent of the drug coating to be delivered to the CNS via tissue of the nasal cavity, in turn, treating central nervous system diseases.

### FIELD OF THE DISCLOSURE

The present disclosure relates generally to the field of medical devices and methods, and more specifically to using intravascular lithotripsy devices and methods for drug delivery beyond the blood-brain barrier.

### BACKGROUND

The technique and treatment of intravascular lithotripsy (IVL) has recently been developed, which is an interventional procedure to modify calcified plaque in diseased arteries. The mechanism of plaque modification is through use of a catheter having one or more acoustic shock wave generating sources located within a liquid that can generate acoustic shock waves that modify the calcified plaque. IVL devices vary in design with respect to the energy source used to generate the acoustic shock waves, with two exemplary energy sources being electrohydraulic generation and laser generation.

For electrohydraulic generation of acoustic shock waves, a conductive solution (e.g., saline) may be contained within an enclosure that surrounds electrodes or can be flushed through a tube that surrounds the electrodes. The calcified plaque modification is achieved by creating acoustic shock waves within the catheter by an electrical discharge across the electrodes. The energy from this electrical discharge enters the surrounding fluid faster than the speed of sound, generating an acoustic shock wave. In addition, the energy creates one or more rapidly expanding and collapsing vapor bubbles that generate secondary shock waves. The shock waves propagate radially outward and modify calcified plaque within the blood vessels. For laser generation of acoustic shock waves, a laser pulse is transmitted into and absorbed by a fluid within the catheter. This absorption process rapidly heats and vaporizes the fluid, thereby generating the rapidly expanding and collapsing vapor bubble, as well as the acoustic shock waves that propagate outward and modify the calcified plaque. The acoustic shock wave intensity is higher if a fluid is chosen that exhibits strong absorption at the laser wavelength that is employed. These examples of IVL devices are not intended to be a comprehensive list of potential energy sources to create IVL shock waves.

More specifically, catheters to deliver IVL therapy have been developed that include pairs of electrodes for electrohydraulically generating shock waves inside an angioplasty balloon. In these devices, the catheter is advanced over a guidewire through a patient's vasculature until it is positioned proximal to and/or aligned with a calcified plaque lesion in a body lumen. The balloon is then inflated with conductive fluid (using a relatively low pressure of 2-4 atm) so that the balloon expands to contact the lesion but is not an inflation pressure that substantively displaces the lesion. Voltage pulses can then be applied across the electrodes of the electrode pairs to produce acoustic shock waves that propagate through the walls of the angioplasty balloon and into the lesions. Alternative devices to deliver IVL therapy can be within a closed volume other than an angioplasty balloon, such as a cap, balloons of variable compliancy, or other enclosure.

In a different realm, treatment of central nervous system diseases (CNSD), such as Parkinson's disease, Alzheimer's disease, epilepsy, and psychiatric disorders, is a challenge yet to be solved due to several confounding factors that restrict uptake in the central nervous system (CNS). Notably, several commonly used drugs exhibit difficulties in evading the blood-brain barrier (BBB), and existing treatment strategies for enhancing delivery through the BBB cause adverse toxic effects on the periphery of the drug delivery site. Additional factors limiting drug uptake in the CNS include first-pass metabolism, slow absorption, fast elimination, and plasma protein binding. Non-invasive therapies, including deep brain stimulation (DBS), spinal cord stimulation (SNS), and oral medications have been investigated to attempt to evade the BBB and increase drug uptake in the CNS, but have been met with limited success. Moreover, surgical treatment of CNSD is often intolerable for patients exhibiting comorbidities and having a limited life expectancy.

A potential alternative pathway to the CNS has been discovered in sinonasal cavities. The sinonasal cavity, which includes the sinuses, nasal cavity, and passages therebetween, contain accessible nerves (e.g., the olfactory nerves) in the vasculature that are connected to the brain, thereby exhibiting a promising pathway for drug molecules to be absorbed into the brain without passing through the BBB. Gel-based drug delivery systems and nasal sprays have been investigated as potential drug transport routes that administer drugs to the brain by delivering the drug intranasally. Gel-based drug delivery systems are highly compatible with a range of drugs, have good solubility, and can be used at high drug concentrations at the desired drug delivery site with reduced systemic side effects. Gels also exhibit desirable biocompatibility properties, are biodegradable, and exhibit sustained drug release over an extended period, thereby enhancing patient compliance. However, the degradation of gels is typically either too fast (e.g., uncross-linked gels) or too slow (e.g., cross-linked gels), thus rendering these systems currently unsuitable for use in nasal drug delivery systems. Furthermore, with reference to nasal spray systems, anatomical differences in the olfactory areas of patients have proven to be a large obstacle in local delivery of a drug using these mechanisms. Thus, it is clear that intranasal drug delivery for the treatment of CNSD holds promise, but a need remains for a robust, efficacious drug delivery system that can overcome the aforementioned existing problems to deliver drugs beyond the blood-brain barrier.

### SUMMARY

Disclosed herein are systems, methods, and devices for using shock waves to evade the blood-brain barrier (BBB) and deliver drugs to the central nervous system (CNS) for treating central nervous system diseases (CNSD). The sinonasal cavity, which contains nerves that are closely connected to the brain, is an example of an anatomical region through which the BBB can be bypassed for drug delivery to the CNS. A catheter configured to generate shock waves can be inserted into the sinonasal cavity, such as a nasal cavity, and controlled to generate shock waves that cause delivery of a therapeutically effective amount of a drug to the central nervous system (CNS) via tissue of the nasal cavity without harming the tissue. The drug can be coated on an outer surface of an enclosure of the catheter that surrounds one or more shock wave emitters of the catheter. Shock wave(s) generated by the one or more shock wave emitters impact the inner surface of the enclosure and cause the drug coated on the outer surface of the enclosure to be ejected from the outer surface and delivered to the tissue of the nasal cavity. Because the drug delivered to the tissue of the nasal cavity bypasses the blood-brain barrier (BBB), in combination with the use of shock waves to enhance drug delivery, the uptake of the drug in the CNS is improved in comparison to existing drug delivery methods, such as oral medications and nasal sprays. Thus, systems, devices, and methods, according to the principles described herein, can improve treatment of CNSD diseases, such as Parkinson's disease, Alzheimer's disease, and epilepsy.

In some aspects, a method for treating a central nervous system disease via a nasal cavity is provided, comprising: advancing a distal portion of a catheter through an intranasal passage to the nasal cavity such that an enclosure of the catheter is positioned at least partially within the nasal cavity, the distal portion of the catheter comprising at least one shock wave emitter that is surrounded by the enclosure, at least a portion of a surface of the enclosure coated with a drug coating; filling the enclosure to expand the enclosure within the nasal cavity; and generating at least one shock wave by the at least one shock wave emitter, the at least one shock wave causing delivery of a therapeutically effective amount of an active agent of the drug coating from the surface of the enclosure to the central nervous system (CNS) via tissue of the nasal cavity.

In some aspects, the method comprises generating a series of shock waves in accordance with a frequency between 1 Hz and 5 Hz. In some aspects, generating the at least one shock wave comprises generating at least one gas bubble within the enclosure that impacts an inner surface of the enclosure to eject at least a portion of the drug coating from the surface of the enclosure. In some aspects, the method comprises delivering one or more voltage pulses to the at least one shock wave emitter to generate the at least one shock wave. In some aspects, the one or more voltage pulses comprises a voltage between 1 kV and 10 kV. In some aspects, the method comprises delivering one or more laser pulses to the at least one shock wave emitter to generate the at least one shock wave. In some aspects, filling the enclosure comprises pressurizing the enclosure to a pressure less than 10 atm. In some aspects, prior to generating the at least one shock wave, the method comprises delivering an irrigation solution to at least one of the intranasal passage and the nasal cavity to clear the at least one of the intranasal passage and the nasal cavity. In some aspects, the irrigation solution comprises saline and/or a drug. In some aspects, the intranasal passage comprises a frontal sinus ostium and the nasal cavity comprises a frontal sinus. In some aspects, generating the at least one shock wave causes the delivery of the active agent of the drug coating to the CNS via olfactory nerves. In some aspects, advancing the distal portion of the catheter through the intranasal passage comprises advancing the catheter over a guidewire. In some aspects, at least a portion of the enclosure is in contact with the tissue of the nasal cavity when generating the at least one shock wave. In some aspects, at least a portion of the enclosure is spaced from the tissue of the nasal cavity when generating the at least one shock wave. In some aspects, the drug coating comprises a crystalline form, an amorphous form, or a combination thereof. In some aspects, the drug coating comprises a plurality of micro-encapsulations containing the active agent. In some aspects, the plurality of micro-encapsulations comprises a plurality of microspheres and/or a plurality of microcapsules comprising a diameter between 0.5 microns and 500 microns.

In some aspects, a device for intranasal drug delivery is provided, comprising: an elongated tube; at least one shock wave emitter configured to generate at least one shock wave; and an enclosure sealed to a distal portion of the elongated tube and surrounding the at least one shock wave emitter, the enclosure fillable with a conductive fluid, and at least a portion of a surface of the enclosure coated with a drug coating, wherein the at least one shock wave is configured to cause delivery of a therapeutically effective amount of an active agent of the drug coating from the surface of the enclosure to a central nervous system (CNS) via tissue of a nasal cavity.

In some aspects, the at least one shock wave emitter is configured to generate the at least one shock wave configured to cause the delivery of the active agent of the drug coating to the CNS via olfactory nerves. In some aspects, the at least one shock wave emitter is configured to generate at least one gas bubble within the enclosure that impacts an inner surface of the enclosure to eject at least a portion of the drug coating from the surface of the enclosure. In some aspects, the at least one shock wave emitter is configured to generate a series of shock waves in accordance with a frequency between 1 Hz and 5 Hz. In some aspects, the enclosure is configured to be pressurized to a pressure less than 10 atm. In some aspects, the elongated tube is configured to deliver an irrigation solution to at least one of an intranasal passage and the nasal cavity to clear the at least one of the intranasal passage and the nasal cavity. In some aspects, the device comprises a guidewire lumen disposed within the elongated tube and configured to receive a guidewire to guide the enclosure through an intranasal passage and to the nasal cavity. In some aspects, the elongated tube comprises a guidewire lumen configured to receive a guidewire and an irrigation lumen configured to deliver an irrigation solution to at least one of an intranasal passage and the nasal cavity. In some aspects, the guidewire lumen is disposed within the irrigation lumen in the elongated tube. In some aspects, the irrigation lumen extends through a length of the enclosure to a fluid outlet at a distal end of the enclosure. In some aspects, the elongated tube is configured to couple to a syringe to fill the enclosure with the conductive fluid. In some aspects, the enclosure is fillable with an x-ray contrast agent to facilitate viewing of the enclosure. In some aspects, the enclosure comprises at least one of an elliptical balloon, spherical balloon, and a hemispherical balloon. In some aspects, at least 20% of the surface of the enclosure is coated with the drug coating. In some aspects, all of the surface of the enclosure is coated with the drug coating. In some aspects, the drug coating comprises a crystalline form, an amorphous form, or a combination thereof. In some aspects, the drug coating comprises a plurality of micro-encapsulations containing the active agent. In some aspects, the plurality of micro-encapsulations comprises a plurality of microspheres and/or a plurality of microcapsules comprising a diameter between 0.5 microns and 500 microns.

In some aspects, a system for intranasal drug delivery is provided, comprising: a device according to any one of the aforementioned examples; and a pulse generator coupled to the at least one shock wave emitter and configured to generate energy pulses to cause the at least one shock wave emitter to generate the at least one shock wave. In some aspects, the pulse generator is configured to generate the energy pulses to cause the at least one shock wave emitter to generate a series of shock waves in accordance with a frequency between 1 Hz and 5 Hz. In some aspects, the pulse generator is configured to generate one or more voltage pulses to cause the at least one shock wave emitter to generate a series of shock waves. In some aspects, the one or more voltage pulses comprises a voltage between 1 kV and 10 kV. In some aspects, the pulse generator is configured to generate one or more laser pulses to cause the at least one shock wave emitter to generate the at least one shock wave. In some aspects, the system comprises an endoscope disposed alongside the device and comprising an imaging sensor at a distal portion of the endoscope configured to capture images of at least one of an intranasal passage and the nasal cavity.

### DESCRIPTION OF THE FIGURES

The invention will now be described, by way of example only, with reference to the accompanying drawings, in which:

FIG. 1A illustrates a distal portion of an exemplary shock wave catheter, according to one or more aspects of the present disclosure.

FIG. 1B illustrates a proximal portion of the exemplary shock wave catheter, according to one or more aspects of the present disclosure.

FIG. 2A illustrates a side view of a distal portion of an exemplary shock wave catheter in a deflated state, according to one or more aspects of the present disclosure.

FIG. 2B illustrates a side view of the distal portion of the exemplary shock wave catheter in an inflated state and emitting shock waves by the shock wave emitters, according to one or more aspects of the present disclosure.

FIG. 2C illustrates a side view of the distal portion of the exemplary shock wave catheter in an inflated state and ejecting the drug from the surface of the enclosure, according to one or more aspects of the present disclosure.

FIG. 3 illustrates a cross-sectional view of an exemplary shock wave catheter, according to one or more aspects of the present disclosure.

FIG. 4 illustrates an exemplary endoscope device for use with the shock wave catheter described herein, according to one or more aspects of the present disclosure.

FIG. 5A illustrates a sagittal slice of the sinonasal cavity with a shock wave catheter disposed in the nasal cavity, according to one or more aspects of the present disclosure.

FIG. 5B illustrates another sagittal slice of the sinonasal cavity with a shock wave catheter disposed in a sinus cavity, according to one or more aspects of the present disclosure.

FIG. 6 illustrates an exemplary method for treating a central nervous system disease (CNSD) via a nasal cavity, according to one or more aspects of the present disclosure.

FIG. 7A illustrates the exemplary shock wave catheter delivering a saline solution to the vascular passageway to clear the vascular passageway, according to one or more aspects of the present disclosure.

FIG. 7B illustrates shock wave emitters of the exemplary shock wave catheter emitting shock waves to cause delivery of the drug through tissue when the shock wave catheter is not in contact with the tissue, according to one or more aspects of the present disclosure.

FIG. 7C illustrates the shock wave emitters of the exemplary shock wave catheter emitting shock waves to cause delivery of the drug through tissue when the shock wave catheter is in contact with the tissue, according to one or more aspects of the present disclosure.

### DETAILED DESCRIPTION

The following description is presented to enable a person of ordinary skill in the art to make and use the various embodiments and aspects thereof disclosed herein. Descriptions of specific devices, assemblies, techniques, and applications are provided only as examples. Various modifications to the examples described herein will be readily apparent to those of ordinary skill in the art, and the general principles described herein may be applied to other examples and applications without departing from the spirit and scope of the various embodiments and aspects thereof. Thus, the various embodiments and aspects thereof are not intended to be limited to the examples described herein and shown but are to be accorded the scope consistent with the claims.

Described herein are systems, methods, and devices for treating central nervous system diseases (CNSD) by delivering a drug using shock waves to anatomical regions that enable access to the central nervous system (CNS) by bypassing the blood-brain barrier (BBB). An exemplary anatomical region for evading the BBB is a sinonasal cavity, the tissue of which includes nerves that enable access to the CNS. A shock wave catheter for use in delivering the drug to the CNS can include at least one shock wave emitter configured to generate shock waves. An enclosure encloses the shock wave emitter(s) and is coated with the drug. The shock wave catheter can be advanced through an intranasal passage to a sinonasal cavity (e.g., a nasal cavity, a sinus cavity) to position the enclosure and shock wave emitter(s) at least partially within the desired sinonasal cavity. The enclosure can be filled with a conductive fluid and one or more voltage pulses can be applied to the at least one shock wave emitter so that shock waves are generated within the conductive fluid. The shock waves can propagate outward from the at least one shock wave emitter and cause the drug coated on the enclosure to be dislodged from the surface of the enclosure and directed onto tissue of the sinonasal cavity. A therapeutically effective amount of the active agent of the drug can be transported by the body into nerves of the central nervous system (CNS), through which it bypasses the BBB and enters into the CNS. In comparison to alternative drug delivery mechanisms (e.g., oral medications, nasal spray), the shock waves can enable delivery of the drug at precisely targeted locations and can cause the drug to propagate deeper through the tissue of the sinonasal cavity, which can result in increased uptake of the drug in the CNS.

The blood-brain barrier (BBB) refers to the protective barriers that separate the blood from the central nervous system structures, including the brain, spinal cord, and eyes. Although the description provided herein describes evading the blood-brain barrier (BBB) for drug delivery to the CNS primarily through the sinonasal cavity, alternative anatomical regions exist in which the BBB is present and could be evaded for drug delivery to the CNS. While there are variations in the barriers at the different anatomical locations (such as blood-spinal cord barrier and blood-ocular barrier), the term blood-brain barrier is used herein as a collective term to encompass these protective barriers within the central nervous system.

As used herein, the term “electrode” refers to an electrically conducting element (typically made of metal) that receives electrical current and subsequently releases the electrical current to another electrically conducting element. In the context of the present disclosure, electrodes are often positioned relative to each other, such as in an arrangement of an inner electrode and an outer electrode. Accordingly, as used herein, the term “electrode pair” refers to two electrodes that are positioned adjacent to each other such that application of a sufficiently high voltage to the electrode pair will cause an electrical current to transmit across the gap (also referred to as a “spark gap”) between the two electrodes (e.g., from an inner electrode to an outer electrode, or vice versa, optionally with the electricity passing through a conductive fluid or gas therebetween). More information about the physics of shock wave generation and their control can be found in U.S. Pat. Nos. 8,956,371, 8,728,091, 9,522,012, and 10,226,265, each of which is incorporated by reference in its entirety. In some contexts, one or more electrode pairs may also be referred to as an electrode assembly. In the context of the present disclosure, the term “emitter” broadly refers to the region of an electrode assembly where the current transmits across the electrode pair, generating a shock wave. The term “emitter band” refers to a continuous or discontinuous band of conductive material that may form one or more electrodes of one or more electrode pairs, thereby forming a location of one or more emitters.

In some embodiments, a shock wave catheter is a so-called “rapid exchange-type” (Rx) catheter provided with an opening portion through which a guidewire is guided (e.g., through a middle portion of a central tube in a longitudinal direction). In other embodiments, a shock wave catheter may be an “over-the-wire-type” (OTW) catheter in which a guidewire lumen is formed throughout the overall length of the catheter, and a guidewire is guided through the proximal end of a hub.

Although shock wave catheters are described herein that generate shock waves based on high voltage applied to electrodes, it should be understood that a shock wave catheter additionally or alternatively may comprise a laser and optical fibers as a shock wave emitter system whereby the laser source delivers energy through an optical fiber and into a fluid to form shock waves and/or cavitation bubbles.

In the following description of the various embodiments, reference is made to the accompanying drawings, in which are shown, by way of illustration, specific embodiments that can be practiced. It is to be understood that other embodiments and examples can be practiced, and changes can be made without departing from the scope of the disclosure.

In addition, it is also to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof. As provided herein, it should be appreciated that any disclosure of a numerical range describing dimensions or measurements such as thickness, length, weight, time, frequency, temperature, voltage, current, angle, etc. is inclusive of any numerical increment or gradient within the ranges set forth relative to the given dimension or measurement.

Exemplary shock wave catheters that can be used for drug delivery, such as by configuring an enclosure (e.g., balloon) with a drug coating, are described in U.S. Pat. Nos. 10,441,300, 11,517,338, and 9,180,280, each of which is incorporated herein by reference in its entirety. Shock wave catheters for treating rhinosinusitis, such as described in U.S. Provisional Patent Application No. 63/456,272, incorporated herein by reference in its entirety, can be configured for drug delivery, such as by a suitable drug coating. Shock wave catheters for drug delivery can be configured to direct shock waves in different directions. For example, forward-biased shock wave catheters, such as that which is described in U.S. Pat. No. 10,966,737 and U.S. Publication No. 2019/0388110, both of which are incorporated herein by reference, direct shock waves in a generally forward direction (e.g., distally from the distal end of a catheter) and can be configured for drug delivery, according to the principles described herein, such as by coating a forward portion of the shock wave with a suitable drug coating. Shock wave catheters configured to generate shock waves emitted from multiple locations that constructively interfere, such as described in U.S. Publication No. 2023/0123003, incorporated herein by reference in its entirety, can be configured for drug delivery, such as by a suitable drug coating. Shock wave catheters configured to deliver several high-voltage pulses in a packet having a short duration (i.e., operable in a “burst mode”), such as described in U.S. patent application Ser. No. 18/595,148, incorporated herein by reference in its entirety, can be configured for drug delivery, such as by a suitable drug coating. Shock wave catheters configured to include arrays of low-profile electrode assemblies that reduce the crossing profile of the catheter and allow the catheter to more easily navigate narrow body lumens, such as described in U.S. Pat. Nos. 8,888,788 and 10,709,462 and U.S. Publication No. 2021/0085347, each of which is incorporated herein by reference in its entirety, can be configured for drug delivery, such as by configuring a suitable drug coating.

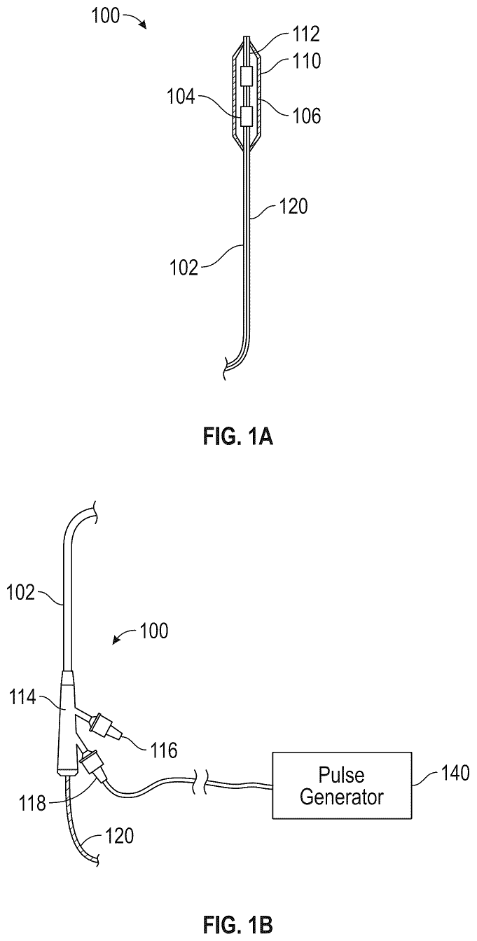

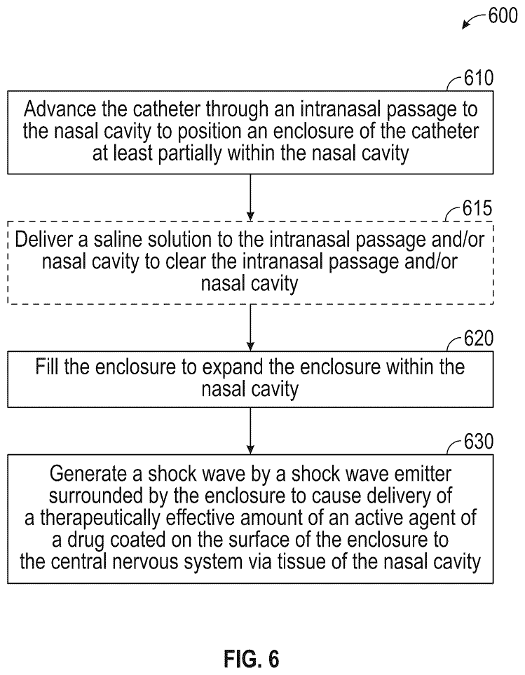

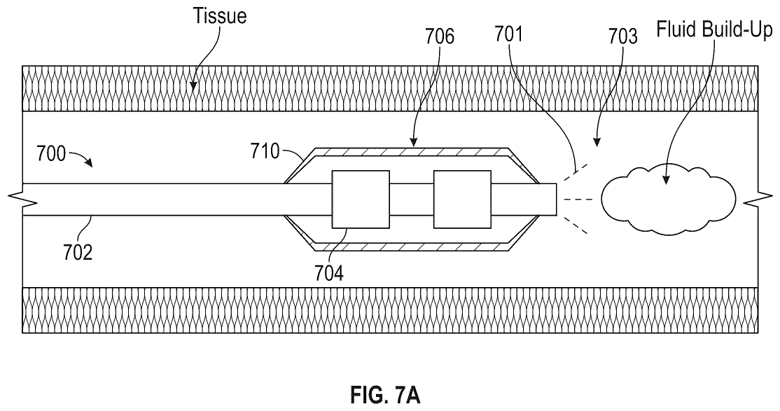

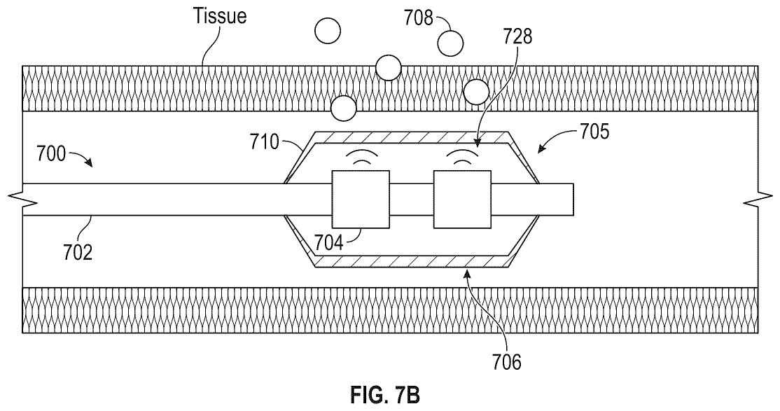

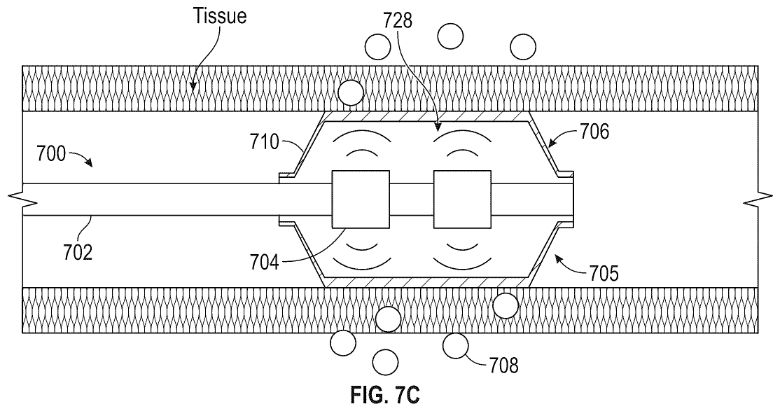

The following description describes exemplary shock wave catheters and methods of use thereof for treating central nervous system diseases (CNSD) with reference to several figures. For example, FIGS. 1A-1B, 2A-2C, 3, and 5A-5B are referenced throughout to describe exemplary shock wave catheters. An example endoscope that may be used with the shock wave catheters described herein is described with respect to FIG. 4. An example method for treating CNSD using the shock wave catheters provided herein is described with respect to the method illustrated in FIG. 6 and the diagrams provided in FIGS. 7A-7C.

### Shock Wave Catheters for Drug Delivery to the CNS

FIGS. 1A-1B illustrate an exemplary shock wave catheter 100 that can be used to deliver a drug to the central nervous system (CNS) by bypassing the blood-brain barrier (BBB). For example, the catheter 100 may be inserted into a sinonasal cavity for delivering the drug to tissue of the sinonasal cavity that includes vasculature for accessing the CNS. In some examples, in addition to or instead of delivering a drug to the CNS, the catheter 100 may be used to deliver shock waves to a targeted region of the CNS for focused ultrasound therapy of that region of the CNS. For example, the catheter 100 may be used to deliver shock waves to the brain, such as for focused ultrasound therapy of the cortex and/or subcortical region of the brain. As illustrated in FIG. 1A, a distal portion 100a of the catheter 100 may include an elongated tube 102, at least one shock wave emitter 104 configured to generate at least one shock wave, and an enclosure 106 sealed to a distal portion of the elongated tube 102 and surrounding the at least one shock wave emitter 104. At least a portion of the outer surface of the enclosure 106 may be coated with a drug coating 110.

In the illustrated example, the catheter 100 includes two shock wave emitters 104. However, catheter 100 can include any number of shock wave emitters, including a single shock wave emitter, greater than two shock wave emitters, at least four shock wave emitters, etc. The catheter 100 may include at least two shock wave emitters 104 positioned adjacent to one another at a sufficiently close distance such that the shock waves generated by the shock wave emitters 104 constructively interfere with one another. In some embodiments, adjacent shock wave emitters may be spaced 4.0 mm or less apart.

The at least one shock wave emitter 104 can be configured to generate a series of shock waves. For example, the at least one shock wave emitter 104 may generate a series of shock waves in accordance with a frequency or duty cycle, such as a frequency between about 1-5 Hz. In some examples, the at least one shock wave emitter 104 may be configured to generate one or more bursts of micro-pulses that are generated in rapid succession (e.g., with a frequency between about 100 Hz-10 kHz). A series of the bursts of micro-pulses can be generated in accordance with the aforementioned frequency between about 1-5 Hz. As described in greater detail below, the frequency of the shock waves generated by the at least one shock wave emitter 104 may be controllable by a pulse generator 140 (illustrated in FIG. 1B) coupled to the shock wave emitter 104. In some examples, the at least one shock wave emitter 104 may generate a series of shock waves in accordance with a frequency greater than or equal to about 1, 2, 3, or 4 Hz. In some examples, the at least one shock wave emitter 104 may generate a series of shock waves in accordance with a frequency less than or equal to about 2, 3, 4, or 5 Hz.

Advantageously, in some embodiments, the properties of the shock waves generated by the at least one shock wave emitter 104 are set or modulated (e.g., by controlling the pulsing or micro-pulsing algorithm, pulse-width, and/or pulse amplitude) to ensure that shock waves do not damage any adjacent cartilage or bony structures. For example, in the case of therapy in the sinonasal cavity, the maximum power delivered to the at least one shock wave emitter may be set to ensure that any pressure waves generated do not damage the bones of the nasal septum.

The enclosure 106 may be attached to the distal portion of the elongated tube 102 via an adhesive or other attachment means. The enclosure 106 may include at least one of an elliptical balloon, a spherical balloon, and a hemispherical balloon. The enclosure 106 may be fillable with a conductive fluid that enables generation of a shock wave from an electrical arc generated by the shock wave emitter 104. The conductive fluid may include water or saline. When a suitable voltage pulse is applied to the shock wave emitter 104, an electrical arc can be formed in the conductive fluid within the enclosure 106. The formation of the electrical arc can create a shock wave that propagates outwardly toward the enclosure 106. In some examples, the enclosure 106 may additionally or alternatively be filled with an x-ray contrast that facilitates viewing of the enclosure 106.

The enclosure 106 may be pressurized to a pressure of less than about 10 atmospheres when the enclosure 106 is filled, such as less than about 5 atm. In some examples, the enclosure 106 is pressurized to a pressure that is sufficient to ensure apposition of at least a portion of the enclosure 106 to the nearby body tissue (e.g., sinonasal cavity). In some examples, the enclosure 106 is pressurized to a pressure up to the enclosure's nominal pressure, which may vary based on the size of the enclosure 106. In some examples, pressurizing the enclosure 106 may not stretch the enclosure 106 itself. In other examples, the enclosure 106 stretches when pressurized to the sufficient pressure noted above. A shock wave generated by the at least one shock wave emitter 104 may cause a pressure spike within the enclosure 106 of less than about 15 atm.

As noted above, the enclosure 106 may be coated with a drug coating 110. At least a portion of the drug coating 110 can be releasable from the surface of the enclosure 106 via the shock waves generated by the at least one shock wave emitter 104. For example, when the at least one shock wave emitter 104 within the enclosure 106 generates at least one shock wave, the shock wave can interact with the enclosure 106 and/or the drug coating 110 itself to cause drug particles from the drug coating 110 to eject from the surface of the enclosure 106. In some examples, shock wave generation is accompanied by the expansion and collapse of a cavitation bubble, which produces one or more micro-jets. These micro-jets may impact the inner surface of the enclosure 106, causing at least a portion of the drug coating 110 to eject from the surface of the enclosure 106.

The drug coating 110 may include a plurality of micro-encapsulations containing the active agent of the drug. For example, the plurality of micro-encapsulations may include a plurality of microspheres and/or a plurality of microcapsules. The shock waves generated by the shock wave emitters 104 may cause the micro-encapsulations to break down into smaller particles and release the active agent held therein. A diameter of the micro-encapsulations may be between about 0.5-500 microns (μm). For example, a diameter of the micro-encapsulations may be between about 0.5-100 μm, 0.5-50 μm, 0.5-10 μm, 5-500 μm, 5-100 μm, −50 μm, 5-20 μm, 10-100 μm, 10-50 μm, 50-500 μm, 50-100 μm, or 100-500 μm. In some examples, a diameter of the micro-encapsulations may be greater than or equal to about 0.5, 1, 2, 5, 10, 20, 50, 100, 150, 200, 250, 300, 350, 400, or 450 μm. In some examples, a diameter of the micro-encapsulations may be less than or equal to about 1, 2, 5, 10, 20, 50, 100, 150, 200, 250, 300, 350, 400, or 450 μm.

The drug coating 110 may include nanoparticles in a crystalline form, an amorphous form, or a combination thereof. For example, the drug coating 110 may include a gel-like structure. As described herein, gel-based drug delivery systems exhibit several desirable characteristics. Gel-based systems are highly compatible with a range of drugs, have good solubility, and can be used at high drug concentrations at the desired drug delivery site with reduced systemic side effects. Gels are also biocompatible, biodegradable, and exhibit sustained drug release over an extended period.

At least a portion of the outer surface of the enclosure 106 may be coated with a drug coating 110. For example, at least about 20% of the surface of the enclosure 106 may be coated with the drug coating 110. In some examples, between about 20-100%, 20-80%, 20-60%, or 20-40% of the surface of the enclosure 106 may be coated with the drug coating 110. In some examples, greater than or equal to about 20%, 30%, 40%, 50%, 60%, 70%, 80%, 90%, or 95% of the surface of the enclosure 106 may be coated with the drug coating 110. In some examples, less than or equal to about 30%, 40%, 50%, 60%, 70%, 80%, 90%, 95%, or 99% of the enclosure 106 may be coated with the drug coating 110.

In some examples, substantially all of the enclosure 106 may be coated with the drug coating 110. In another example, at least the distal portion of the enclosure 106 may be coated with the drug coating 110. In another example, a central portion of the enclosure 106 may be coated with the drug coating 110. In another example, a proximal portion of the enclosure 106 may be coated with the drug coating 110. Any combination of the aforementioned coating patterns is understood to be encompassed by the scope of this disclosure. For example, the central portion and distal portion of the enclosure 106 may be coated with the drug coating 110.

The drug coating 110 may be applied to the enclosure 106 by coating, brushing, dipping, spraying, and/or soaking the enclosure 106 with a fluid (e.g., a gas or liquid) including the drug. In some examples, after applying the drug coating 110 to the enclosure 106, the enclosure 106 may be air-dried, heat-treated, or cooled to allow the drug coating 110 to sufficiently adhere to the surface of the enclosure 106. In this manner, the drug coating 110 can be selectively applied to only a portion of the enclosure 106, as desired.

The elongated tube 102 of the catheter 100 may include one or more lumens 112. The one or more lumens 112 can include a lumen for filling the enclosure 106 (e.g., with conductive fluid). The one or more lumens 112 can include a lumen for delivering an irrigation solution to the anatomical region that the catheter 100 is inserted to, such as a sinonasal cavity. In this example, the irrigation solution can be used to clear the sinonasal cavity of obstruction (e.g., mucus) in preparation for the drug delivery. The irrigation solution may include saline. In some examples, the irrigation solution may include a drug, such as a hydrophilic drug. The irrigation lumen can extend through a length of the catheter 100 to a distal end of the catheter 100. During use of catheter 100, the irrigation solution can be introduced to the lumen at a proximal end of the catheter 100, and can be ejected (e.g., sprayed) from the distal end of the catheter 100 into the sinonasal cavity. Once the cavity is cleared, the catheter 100 can be used for drug delivery in the sinonasal cavity.

The enclosure 106 of catheter 100 may be positioned in the desired region of the body using a guidewire. FIG. 1B illustrates a guidewire 120 extending from a proximal end of the catheter 100. In the instance where the catheter 100 is used with a guidewire 120, the one or more lumens 112 may include a lumen configured to receive a guidewire (i.e., a guidewire lumen).

The elongated tube 102 may be manufactured from compliant materials and/or may be configured with particular geometries that enable the elongated tube 102 to be torqued, curved, and physically manipulated to maneuver the catheter 100 to the appropriate treatment region within the body. For example, a portion of the elongated tube 102 can include slits, a coiled region, or other cut-outs that enable a user to maneuver the catheter 100.

The dimensions (e.g., length, diameters, etc.) of different embodiments of the catheter 100 may be selected for targeting different patient anatomies. For example, a length of the elongated tube 102, i.e., the distance from a distal end of the elongated tube 102 (inclusive of the length of the enclosure 106) to the proximal end of the elongated tube 102 proximate to the handle 114, may be between about 10-200 cm. In some examples, the length of the elongated tube 102 may be between about 20-100 cm or 40-80 cm.

As noted above and illustrated in FIG. 1B, the catheter 100 may include a handle 114 at the proximal end of the elongated tube 102. The handle 114 may facilitate connection between one or more fluid sources and one or more lumens 112 of the elongated tube 102 at one or more fluid ports 116. The handle 114 may facilitate connection between one or more electrical components (e.g., pulse generator 140) and wires disposed within the elongated tube 102 via one or more electrical ports 118. In examples in which the catheter 100 is usable with a guidewire 120, the handle 114 may be configured to receive the guidewire 120 and provide the guidewire 120 to a corresponding lumen of the elongated tube 102. The handle 114 may be configured such that the user can grasp the handle 114 to manipulate the catheter 100. Using the handle 114, the user may operate the catheter 100 with just one hand. In some examples, the handle 114 may include one or more controls (e.g., buttons, switches, knobs, etc.) for controlling operation of the catheter 100. For example, the handle 114 may include one or more controls for controlling fluid flow into and/or out of the enclosure 106 (e.g., via a lumen of elongated tube 102). The handle 114 may include one or more controls for controlling delivery of energy to the one or more shock wave emitters 104 (e.g., via electrical wires contained within elongated tube 102, described in greater detail below). The handle 114 may include one or more controls for controlling delivery of an irrigation solution through an irrigation lumen of the elongated tube 102 and out of the distal end of the elongated tube 102.

As noted above, the handle 114 may include one or more fluid ports 116 configured to receive fluid from an external fluid source and deliver the fluid to a corresponding lumen of elongated tube 102. The one or more fluid ports 116 may be configured to couple to different fluid sources. For example, the one or more fluid ports 116 may removably couple to a syringe or pump including a conductive fluid (e.g., saline) to fill the enclosure 106 with the conductive fluid. The one or more fluid ports 116 may removably couple to a syringe or pump including an irrigation solution to deliver the irrigation solution to the desired anatomical region (e.g., a sinonasal cavity) via the elongated tube 102. The one or more fluid ports 116 may be configured to receive fluid from the elongated tube 102 to remove the fluid from the catheter 100 and may dispose the fluid in a fluid reservoir removably connected to the one or more fluid ports 116. The one or more fluid ports 116 may removably couple to fluid sources and/or fluid reservoirs using connectors (e.g., quick disconnect connectors, Luer connectors, threaded connectors, etc.) and/or tubing.

As illustrated in FIG. 1B and mentioned above, the catheter 100 may be electrically coupled (e.g., via wired or wireless communication) to a pulse generator 140. For example, the catheter 100 may be electrically coupled to a pulse generator 140 via one or more cables and an electrical port 118. One or more wires may extend from the pulse generator 140, through the electrical port 118, and through the elongated tube 102 to transmit energy from the pulse generator 140 and to the at least one shock wave emitter 104. The wires may include copper or another conductive material that can transmit electrical energy. The wires may be insulated by an insulation material to protect the wires from damage and wear over time, both within the elongated tube 102 and external to the catheter 100.

The pulse generator 140 may be configured to generate energy pulses (e.g., voltage, laser) to cause the at least one shock wave emitter 104 to generate at least one shock wave. For example, voltage pulses generated by the pulse generator 140 may be delivered to the at least one shock wave emitter 104 to cause the shock wave emitter 104 to generate at least one shock wave based on the voltage pulses. The voltage pulses may include a voltage between 1-10 kV. In some examples, the voltage pulses may include a voltage between about 1-8 kV, 1-5 kV, 5-8 kV, or 5-10 kV. The voltage pulses may include a voltage greater than or equal to about 1, 2, 5, or 8 kV. The voltage pulses may include a voltage less than or equal to about 2, 5, 8, or 10 kV.

The pulse generator 140 may be configured to control at least one of the amplitude, pulse width, frequency, and duty cycle of the energy pulses applied across the electrodes of the shock wave emitter 104. For example, based on the energy pulse(s) delivered to the at least one shock wave emitter 104 by the pulse generator 140, the at least one shock wave emitter 104 may be configured to generate a series of shock waves in accordance with a duty cycle or frequency, such as a frequency between about 1-5 Hz. In some examples, the at least one shock wave emitter 104 may generate a series of shock waves in accordance with a frequency greater than or equal to about 1, 2, 3, or 4 Hz. In some examples, the at least one shock wave emitter 104 may generate a series of shock waves in accordance with a frequency less than or equal to about 2, 3, 4, or 5 Hz. In some examples, the pulse generator 140 may be configured to deliver a packet of micro-pulses having a frequency between about 100 Hz-10 kHz. The pulse generator 140 may deliver a series of the packets of micro-pulses in accordance with the aforementioned frequency between about 1-5 Hz. In some examples, the amplitude of the energy pulses may be gradually increased (or decreased) over the duration of the procedure to cause release of the drug particles from the surface of the enclosure.

In an alternative example, the pulse generator 140 may include a laser source configured to deliver energy to the at least one shock wave emitter 104 via optical fiber(s). The pulse generator 140 may be configured to deliver laser pulses to the at least one shock wave emitter 104 to cause the shock wave emitter 104 to generate at least one shock wave.

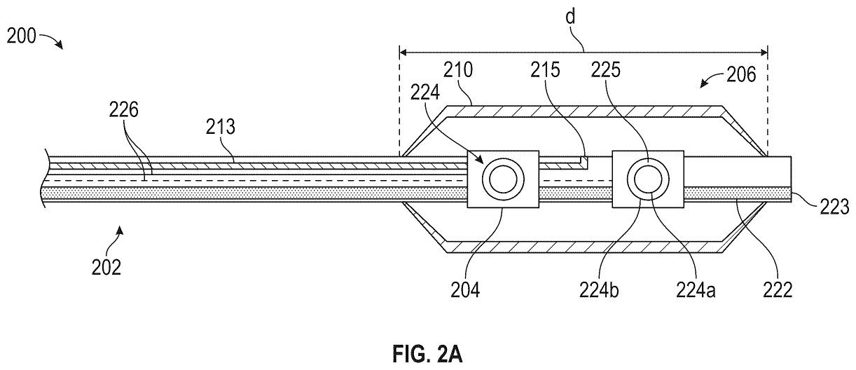

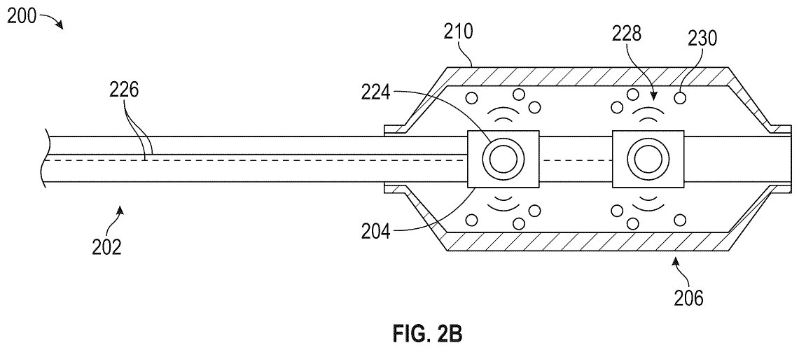

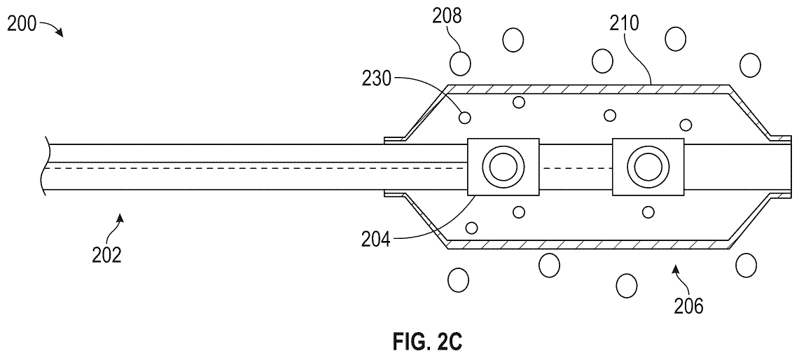

As mentioned above, the shock wave catheters described herein can include one or more lumens for filling the enclosure and/or delivering an irrigation solution to the desired anatomical region, such as a sinonasal cavity. FIG. 2A illustrates a side view of a shock wave catheter 200 that includes at least two lumens for these purposes. The catheter 200 can include any one or more features of catheter 100 described herein with respect to FIGS. 1A-1B.

The elongated tube 202 can include a lumen 213 for filling the enclosure 206. The lumen 213 may include at least one fluid outlet 215 that fluidly connects the inner region of the enclosure 206 and the lumen 213. Although a single fluid outlet 215 is illustrated in FIG. 2A, the lumen 213 may include a plurality of fluid outlets 215 along the length of the elongated tube 202 that is surrounded by the enclosure 206 (e.g., 2, 3, 4, or more fluid outlets 215).

The elongated tube 202 can include an irrigation lumen 222 for delivering an irrigation solution to the anatomical region in which at least the distal portion of the catheter 200 is inserted. The lumen 222 can extend through a length of the elongated tube 202, including through a length of the enclosure 206 to a fluid outlet 223 at the distal end of the elongated tube 202.

As mentioned above, the shock wave catheters described herein can include at least one shock wave emitter. The example catheter 200 illustrated in FIG. 2A includes two shock wave emitters 204. Each shock wave emitter 204 can include an electrode assembly 224, otherwise referred to herein as an electrode pair. The electrodes of the electrode assembly 224 can be positioned adjacent to one another. For example, the electrode assembly 224 can include an inner electrode 224a surrounded by an outer electrode 224b, with a gap 225 between the two electrodes. In another example, the electrodes of the electrode assembly 224 can be placed side by side or in another arrangement that includes the gap 225 between the two electrodes. When the enclosure 206 is filled with a fluid and a suitable voltage pulse is applied across the electrode assembly 224, a spark forms at the fluid-filled gap 225 between the electrodes of the electrode assembly 224. The spark causes generation of a cavitation bubble at the shock wave emitter 204 that rapidly expands and collapses, in turn causing propagation of a shock wave from the shock wave emitter 204.

The electrodes of the electrode assembly 224 can include a conductive material. For example, an emitter band of a conductive material may be disposed on the elongated tube 202 to form one or more electrodes of the electrode assembly 224. In some examples, an end of a conductive wire extending within the elongated tube 202 to the shock wave emitter 204 may form an electrode of the electrode assembly 224.

As mentioned above, the shock wave emitter(s) of the shock wave catheters described herein (e.g., catheter 200) can be coupled to a pulse generator to deliver energy to the shock wave emitter(s). The example catheter 200 illustrated in FIG. 2A includes electrical wires 226 to electrically couple each shock wave emitter 204 (i.e., the electrode assembly 224 of the shock wave emitter 204) to a pulse generator. The wires 226 can extend within the elongated tube 202 to the shock wave emitters 204. In some examples, the wires 226 can extend within a dedicated lumen within the elongated tube 202.

The enclosure 206 may be manufactured from one or more compliant or semi-compliant (e.g., elastic, flexible) materials. For example, the enclosure 206 may be made from at least one of nylon, polyamide, polyether copolymer, polyurethane, another polymer, or a mixture thereof. The enclosure 206 can include a layered composition, in which each layer includes at least one of the aforementioned materials. In a non-limiting example, the enclosure 206 may be composed of an inner layer including polyamide and an outer layer including nylon, or vice versa. In another non-limiting example, the enclosure 206 may be composed of an inner layer including polyurethane and an outer layer including polyamide, or vice versa. The enclosure 206 may include more than two layers. In a non-limiting example, the enclosure 206 may be composed of an inner layer including polyamide, a middle layer including polyamide (i.e., the same or a different composition of polyamide), and an outer layer including nylon, or vice versa. In another example, the enclosure 206 may be composed of an inner layer including polyamide, a middle layer including polyurethane, and an outer layer including polyamide, or vice versa.

The dimensions (e.g., length, diameters, etc.) of the enclosure 206 may be variable based on patient anatomies. For example, a length of the enclosure 206 may be between about 10-1000 mm, 10-500 mm, or 20-100 mm. As illustrated in FIG. 2A, the length of the enclosure 206 extends between the tapered ends of the enclosure 106 and is denoted as “d”.

A diameter of the enclosure 206 when fully filled may be between about 3-40 mm, 10-40 mm, or 20-40 mm. A combination of dimensions (e.g., diameter and length) of the enclosure 206 may be selected by a physician based on characteristics of the patient, such as age, gender, disease, anatomical restrictions, etc.

FIGS. 2A-2C illustrate an example operation sequence for releasing a drug from the enclosure 206 of catheter 200 using shock waves. FIG. 2A illustrates the catheter 200 prior to shock wave generation with the enclosure 206 in an unfilled state. The enclosure 206 can be unfilled when positioning the enclosure 206 of the catheter 200 in the desired anatomical region for treatment, such as a body lumen or cavity. The enclosure 206 includes a drug coating 210 thereon.

FIG. 2B illustrates the at least one shock wave emitter 204 emitting shock waves 228. The shock waves 228 can propagate outward to the enclosure 206. In some examples, the shock waves generate one or more micro-jets 230. The micro-jets 230 generated by the shock waves can propagate outward from the at least one shock wave emitter 204 toward the inner surface of the enclosure 206 and can impact the inner surface of the enclosure 206.

FIG. 2C illustrates drug particles 208 of the drug coating 210 being ejected from the surface of the enclosure 206, as a result of the shock waves 228 and/or micro-jets 230. In some examples, the enclosure 206 can be placed within a body lumen or cavity such that the enclosure 206 contacts tissue of the body lumen/cavity. In this example, the generated shock waves 228 and/or micro-jets 230 can cause the drug particles 208 to dislodge from the surface of the enclosure 206 and adhere to the tissue to be absorbed into the tissue. In some examples, the enclosure 206 can be placed proximate to the tissue with a space between the enclosure 206 and the tissue. In this example, the shock waves 228 and/or micro-jets 230 can cause the drug particles 208 to eject from the surface of the enclosure 206, and the shock waves 228 may further assist in the travel of the drug particles 208 within the space between the enclosure 206 and the tissue. When the drug particles reach the tissue, they may adhere to the tissue to be absorbed into the tissue.

In some examples, the shock waves 228 and/or micro-jets 230 can cause the drug particles 208 to break down into smaller particle sizes. Smaller drug particles can enable the drug particles 208 to be more easily ejected from the surface of the enclosure 206 and/or propelled from the surface of the enclosure 206 and to the tissue by the shock waves 228.

In some examples, the shock waves 228 can cause the drug particles 208 to embed within the tissue. In some examples, the shock waves 228 can enable the drug particles 208 to propagate through the tissue of the body lumen or cavity (e.g., a sinonasal cavity) and into the CNS. The shock waves 228 may drive the drug particles 208 deeper through the tissue, for example, as compared to a drug uptake mechanism that solely relies on the anatomy of the sinonasal cavity itself to drive the drug particles 208 through the tissue and into the desired location of the CNS. Deeper propagation of the drug particles 208 may increase uptake of the active agent within the drug particles 208 in the CNS.

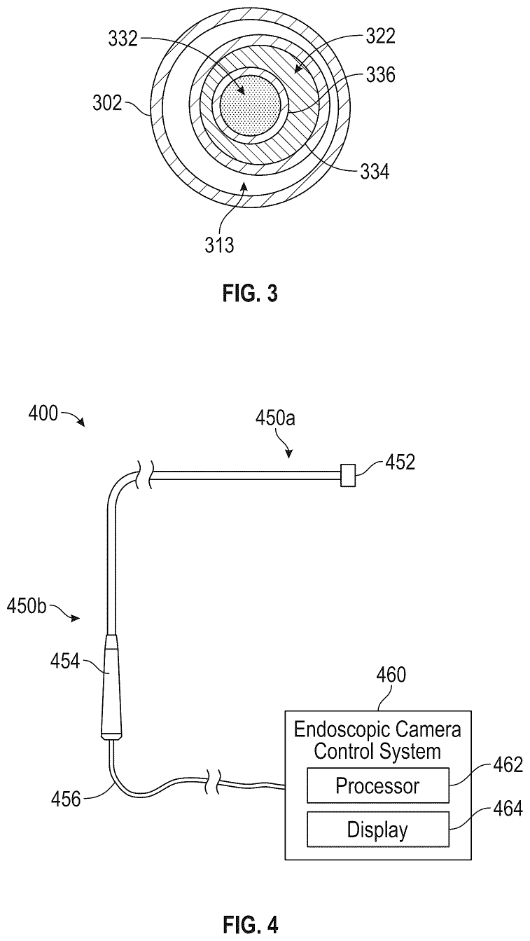

As noted above, the elongated tube 202 may include one or more lumens. FIG. 3 illustrates a cross-sectional view of an elongated tube 302 surrounding several lumens. The elongated tube 302 can be used for one or more of elongated tubes 102, 202 of FIGS. 1A-1B and 2A-2C, respectively. The elongated tube 302 includes a lumen 313 for filling an enclosure of a catheter, an irrigation lumen 322 for delivering an irrigation solution to the lumen or cavity of a desired anatomical region, and a guidewire lumen 332 that receives a guidewire.

The lumen 313 may be the innate lumen of the elongated tube 302. Each of the irrigation lumen 322 and guidewire lumen 332 may also be a lumen of a tube (e.g., tube 334 and tube 336, respectively). The guidewire lumen 332 may be disposed within the irrigation lumen 322. Each of the guidewire lumen 332 and/or the irrigation lumen 322 may be disposed within the lumen 313 of the elongated tube 302. Alternative arrangements of the lumens of the elongated tube 302 are also envisaged. For example, the irrigation lumen 322 may be disposed within the guidewire lumen 332. In some examples, the elongated tube 302 may include a lumen (e.g., lumen 313) configured to receive each of the guidewire and fluid for filling the enclosure. In this example, the irrigation lumen 332 may be disposed within said lumen 313.

As mentioned above, the shock wave catheters (e.g., catheters 100, 200) may be operable alongside an endoscope for viewing a cavity, lumen, or other passage of the body. FIG. 4 illustrates an endoscopic imaging system 400 that may be used with the shock wave catheters described herein for viewing body lumens/cavities. The endoscopic imaging system 400 can include an endoscope 450 for insertion into the body along with the catheter (e.g., catheters 100, 200) for capturing imaging data of the body lumen/cavity. The endoscope 450 can include an imaging sensor 452, which can be positioned at a distal portion 450a of the endoscope 450 or at a proximal portion 450b of the endoscope 450. The imaging sensor 452 is configured to capture images and/or video of the body lumen, cavity, and passages therein. For example, the endoscope 450 may be inserted to a sinonasal cavity, and the imaging sensor 452 may capture images/video of intranasal passages, a nasal cavity, a sinus, etc.

In some examples, an imaging sensor may additionally or alternatively be integrated into the shock wave catheter itself. For example, at least one imaging sensor may be disposed along the elongated tube, within or proximate to the enclosure. Alternatively, a guidewire that can be received by the shock wave catheters described herein may include an imaging sensor (e.g., at a distal end of the guidewire).

The imaging sensor 452 of the endoscope 450 can be communicatively coupled (e.g., via a wired or wireless connection) to an endoscopic camera control system 460. For example, as illustrated in FIG. 4, the endoscope 450 and the endoscopic camera control system 460 may be communicatively coupled via a wired connection 456. The endoscopic camera control system 460 may include a processor 462 and a display 464. The processor 462 may be configured to process imaging data captured by the imaging sensor 452. The processor 462 may be configured to transmit the processed imaging data to the display 464 for display of one or more images, videos, etc. (collectively referred to herein as media) on the display 464 during a shock wave treatment. Using the displayed media, the catheter (e.g., catheters 100, 200) can be positioned at the appropriate treatment site.

In some examples, the endoscopic imaging system 400 can include an illumination system for illuminating the body lumen/cavity during endoscopic imaging. The illumination system may include one or more light emitting diodes (LEDs), for example, disposed along and/or at a distal end of the endoscope 450. In some examples, the illumination system may include an illuminator external to the endoscope, the illuminator coupled to the endoscope via a cable for delivering light to the region imaged with the endoscope.

In some examples, the shock wave catheter (e.g., catheters 100, 200) can include one or more location sensors configured to detect and transmit location data to a processor (e.g., processor 462). For example, a distal portion of the catheter can include one or more location sensors. The processor 462 can process the data to track the movement of the distal portion of the catheter as it is advanced and positioned within the body.

### Methods for Treating Central Nervous System (CNS) Diseases Using Shock Wave Catheters

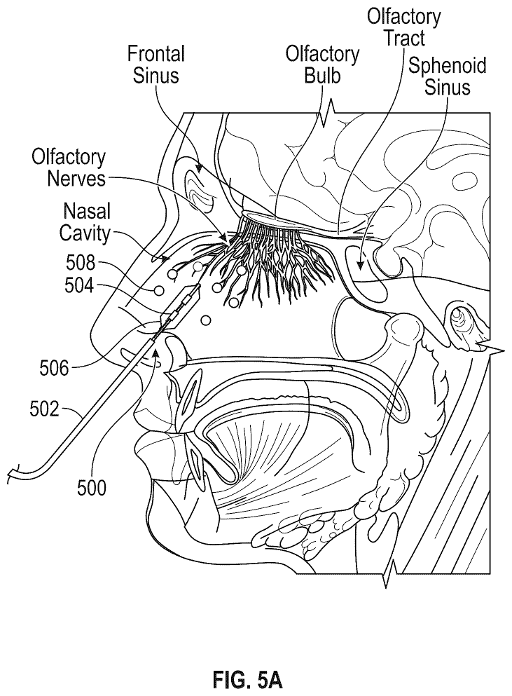

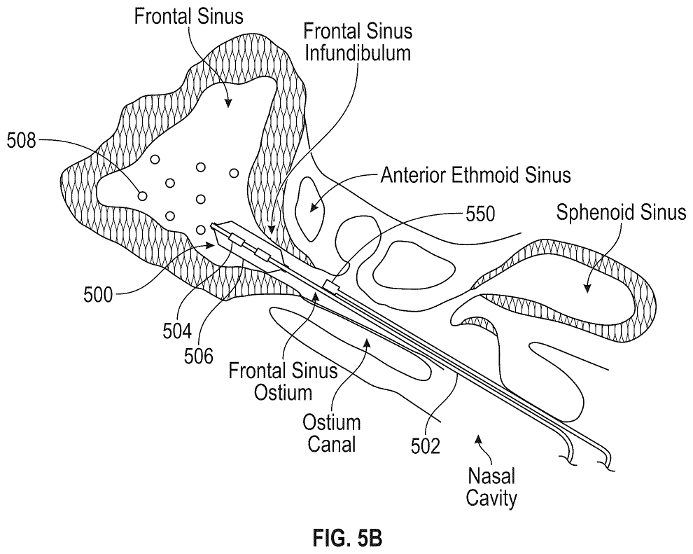

As noted above, the shock wave catheter (e.g., catheters 100, 200) can be disposed within a sinonasal cavity for delivering a drug coated on the enclosure of the catheter into the CNS via tissue of the sinonasal cavity. The sinonasal cavity may include a nasal cavity, sinus cavity, intranasal passage, etc. FIG. 5A illustrates the use of an exemplary catheter 500 in a nasal cavity for delivery of a drug into the CNS via tissue of the sinonasal cavity. The catheter 500 can include any one or more features described herein with respect to catheters 100, 200, illustrated in FIGS. 1A-1B, 2A-2C and described in greater detail above.

The catheter 500 may be positioned within the nasal cavity to deliver a drug to the CNS. The catheter 500 may be positioned within the nasal cavity via an intranasal passage, such as the nostril. The nasal cavity may be selected for sinonasal drug delivery because the olfactory nerves, which lead to the brain via the olfactory bulb and olfactory tract, are accessible. Once the enclosure 506 of the catheter 500 is properly positioned within the nasal cavity, at least one shock wave can be generated by at least one shock wave emitter 504 of the catheter 500 to cause drug particles 508 to eject from the surface coating of the enclosure 506. When ejected proximate to the olfactory nerves, as illustrated, the drug particles 508 can be absorbed into the tissue of the nasal cavity and travel to the CNS, beginning with the olfactory nerves. Thus, the shock waves generated by the shock wave emitter 504 can ultimately result in delivery of the drug (e.g., the active agent of the drug) to the brain by causing the active agent to be delivered to the tissue of the nasal cavity, wherein it may be absorbed into the tissue, move into the olfactory nerves, travel to the olfactory bulb, and travel to the brain via the olfactory tract.

The catheter 500 may be positioned in areas of the sinonasal cavity other than the nasal cavity, such as in a sinus cavity, by advancing the catheter 500 through an intranasal passage of the sinonasal cavity and to the sinus cavity. For example, as illustrated in FIG. 5B, the catheter 500 may be advanced to a position that is at least partially in a sinus cavity. In this illustrated example, a distal portion of the enclosure 506 is positioned within the frontal sinus, whereas the remaining enclosure 506 is positioned within the frontal sinus infundibulum. The intranasal passage connecting the frontal sinus to the nasal cavity can include the frontal sinus ostium and the ostium canal. Although not explicitly illustrated, it is to be understood that substantially all of the enclosure 506 may be positioned within the sinus cavity (e.g., the frontal sinus) or the intranasal passage (e.g., the frontal sinus ostium, the ostium canal). In some examples, the catheter 500 may be advanced through the intranasal passage and positioned at least partially within the anterior ethmoid sinus or the sphenoid sinus. From tissue of any of the above-listed sinus cavities and intranasal passageways, the shock waves generated by the catheter 500 can cause drug delivery to the CNS, for example via the olfactory nerves, olfactory bulb, and olfactory tract.

As noted above, the shock wave catheters described herein can be operated with an endoscope. FIG. 5B illustrates a catheter 500 used alongside an endoscope 550 for viewing a sinonasal cavity (including intranasal passages therein). The endoscope 550 can be used during placement of the catheter 500 and/or during drug delivery via the catheter 500. Although not explicitly illustrated in FIGS. 5A-5B, placement of the catheter 500 may additionally or alternatively include the use of a guidewire extending through the catheter, described above at least with respect to FIGS. 1A-1B and FIG. 3.

FIG. 6 illustrates an exemplary method for treating CNSD using a shock wave catheter, such as catheters 100, 200, and 500 described with respect to FIGS. 1A-1B, 2A-2C, and 5A-5B, respectively.

At block 610 of method 600 in FIG. 6, a distal portion of a catheter is advanced through an intranasal passage to a nasal cavity such that the enclosure of the catheter is positioned at least partially within the nasal cavity. The enclosure of the catheter can be in a deflated (e.g., unfilled) state when advanced through the sinonasal cavity. In some examples, advancing the distal portion of the catheter through the intranasal passage can include advancing the catheter over a guidewire.

At block 615 of method 600 in FIG. 6, an irrigation solution may be delivered to the intranasal passage and/or the nasal cavity by the catheter to clear the intranasal passage and/or the nasal cavity. The irrigating solution can include a saline solution. An example of irrigating an intranasal passage with the catheter is illustrated in FIG. 7A, in which a catheter 700 is used to deliver an irrigation solution 701 to an intranasal passage 703. The catheter 700 illustrated in FIGS. 7A-7C can include any one or more features of catheters 100, 200, and 500 described herein with respect to FIGS. 1A-1B, 2A-2C, and 5A-5B, respectively. For example, the catheter 700 can include an irrigation lumen that extends through the length of the elongated tube 702 and exits at the distal end of the catheter 700.

At block 620 of method 600 in FIG. 6, the enclosure of the catheter can be filled to expand the enclosure within the nasal cavity. FIG. 7B illustrates an expanded (filled) enclosure 706 within a nasal cavity 705. Filling the enclosure 706 can include pressurizing the enclosure to a pressure less than about 10 atm. The enclosure 706 can be filled with a conductive fluid, such as saline. In some examples, the enclosure 706 can additionally or alternatively be filled with an x-ray contrast agents for viewing of the enclosure 706.

At block 630 of method 600 in FIG. 6, a shock wave may be generated by at least one shock wave emitter of the catheter to cause delivery of a therapeutically effective amount of an active agent from the drug coating to the CNS via tissue of the nasal cavity. One or more shock waves can be generated when a suitable energy (e.g., voltage, laser) pulse is applied to an electrode assembly of the at least one shock wave emitter. For example, one or more shock waves may be generated by a spark formed at a fluid-filled gap between the electrodes of an electrode assembly. The shock wave(s) can cause particles from the drug coating to dislodge from the surface of the enclosure. FIGS. 7B-7C illustrate shock waves 728 being generated at shock wave emitters 704. The drug particles 708 dislodged from the surface of the enclosure 706 may deposit on the tissue of the nasal cavity 705. The drug coating 710 may be dislodged from the surface of the enclosure 706 due to the pressure increase in the enclosure caused by the shock wave(s) 728. In some examples, one or more shock waves generate micro-jets within the enclosure that impact the inner surface of the enclosure and cause the drug particles to be dislodged from the surface of the enclosure.

The method 600 can include generating a series of shock waves in accordance with a set frequency or duty cycle. For example, the frequency may be between about 1-5 Hz, such as about 1, 2, 3, 4, or 5 Hz. In some examples, the method 600 can include generating one or more bursts of micro-pulses. The micro-pulses may be generated in rapid succession, for example, in accordance with a frequency of about 100 Hz-10 kHz. Several bursts may be generated in accordance with the aforementioned frequency between about 1-5 Hz. The series of shock waves can drive the ejected drug particles of the drug coating through the tissue and into the CNS. The pulse duration of the energy pulses applied to the electrodes of the shock wave emitter can be dependent on the surface area of the electrodes. For example, the pulse duration can be on the magnitude of a few microseconds (e.g., between about 1-5 μs).

As noted above, delivering the drug to the CNS via a sinonasal cavity can include contacting tissue of the sinonasal cavity with the enclosure or spacing at least a portion of the enclosure from the tissue of the sinonasal cavity. In FIG. 7B, the enclosure 706 is illustrated as being spaced from the tissue of the nasal cavity 705 when generating and emitting the shock wave 728. The minimum distance between the tissue of the nasal cavity 705 and the enclosure 706 may be dependent on the amplitude, direction, frequency, etc. of the shock wave generated by the shock wave emitters therein. FIG. 7C illustrates an alternative arrangement of the enclosure 706 of the catheter 700 within the nasal cavity 705. In FIG. 7C, at least a portion of the enclosure 706 can be placed in contact with the tissue of the nasal cavity when generating and emitting the shock wave 728. For example, at least about 25%, 50%, 75%, or substantially all of the enclosure 706 can be in contact with the tissue of the nasal cavity 705. In some examples, the enclosure 706 may be in contact with the tissue at a first side of the enclosure 706, but may be spaced from the tissue (e.g., by an amount described above) at a second side of the enclosure 706 opposite the first side. In another example, the enclosure 706 can be in contact with the tissue at a distal end of the enclosure, and may be spaced from the tissue (e.g., by an amount described above) at a proximal end of the enclosure 706, or vice versa.

In some instances, the method 600 can include generating and delivering a shock wave simultaneously or near simultaneously with delivering the irrigation solution to the sinonasal cavity (e.g., via an intranasal passage, sinus cavity, and/or nasal cavity). In this manner, excess fluid (e.g., mucus) can be drained from the sinonasal cavity at about the same time as drug release from the surface of the enclosure is initiated.

Although the method 600 primarily describes drug delivery to the CNS via a sinonasal cavity, the methods described herein are not intended to be limited to this anatomical region of the body. For example, the shock wave catheters described herein may be insertable to alternative body lumens and cavities through which the CNS may be accessible. For example, shock wave catheters described herein may be insertable to cavities and/or lumens of or proximate to the eye and the spinal cord for delivering a drug to the CNS by bypassing the BBB in these regions.

The methods described herein may be used to treat various central nervous system diseases (CNSD). CNSD that can be treated using the methods described herein may include but are not limited to: Parkinson's disease, Alzheimer's disease, Huntington's disease, epilepsy, schizophrenia, brain cancer, lung cancer, tracheal cancer, prostate cancer, encephalomyelitis, seizures, myasthenia gravis, atherosclerosis, sclerosis multiplex, amyloid angiopathy, ischemic injury, spinocerebellar ataxia type 1, kainic-acid induced apoptosis (KAIA), hypoxia-induced oxidative stress, tauopathy, hyperactivity, sleep deprivation, obesity, eating disorders, depression, anxiety, autism, addictions, and/or stroke.

The active agent of the drug coating may be dependent on the CNSD being treating using the shock wave catheters and methods provided herein. Example active agents may include, but are not limited to, any one or more of the following: tyrosine kinase inhibitor, NAP peptide, [Ser(2)]exendin (1-9), pituitary adenylate cyclase-activating polypeptide, recombinant human nerve growth factor, L-DOPA, insulin-like growth factor 1, 22C4 single-chain variable fragment antibody, erythropoietin+insulin-like growth factor 1, nerve growth factor, vascular endothelial growth factor, transforming growth factor β1, fibroblast growth factor, mesenchymal stem cells, galanin-like peptide, leptin, interferon β-1b, apolipoprotein B-100, fusion protein mCTA1-T146, herpes simplex virus type 2 ΔRR+ICP10PK and/or filamentous bacteriophage+myelin oligodendrocyte glycoprotein. Additional active agents for treating CNSD will be apparent to one of ordinary skill in the art and are understood to be encompassed by the disclosures provided herein.

In some examples, in addition to or instead of using the shock wave catheters described herein to deliver a drug to the CNS, the shock wave catheters may be used for focused ultrasound therapy of a particular region of the CNS using shock waves. For example, the shock wave catheters described herein may be used to deliver shock waves to the brain, such as to the cortex and/or subcortical region of the brain.

As used herein, the term “inflating” the enclosure is intended to mean introducing fluid to the enclosure, thereby increasing the fluid pressure within the enclosure. Likewise, “deflating” the enclosure is intended to mean removing fluid from the enclosure, thereby decreasing the fluid pressure within the enclosure. Inflating and deflating the enclosure is not intended to connote causing the material forming the enclosure to undergo elastic stretching (or shrinking) as the fluid pressure within the enclosure changes.

Although the electrode assemblies and catheters described herein have been discussed primarily in the context of drug delivery to the CNS (central nervous system) via sinonasal cavities, the electrode assemblies and catheters herein can be used for a drug delivery elsewhere in the vascular system that may provide access to the CNS. For further examples, similar catheter designs may be used for pharmacologic treatment of soft tissues, such as cancer and tumors, blood clots, fibroids, cysts, organs, and scar and fibrotic tissue removal. Electrode assembly and catheter designs could also be used for neurostimulation treatments, targeted drug delivery, treatments of tumors in body lumens (e.g., tumors in blood vessels, the esophagus, intestines, stomach, or vagina), wound treatment, non-surgical removal and destruction of tissue, or instead of thermal treatments or cauterization for venous insufficiency and fallopian ligation (i.e., for permanent female contraception).

In one or more examples, the electrode assemblies and catheters described herein could also be used for tissue engineering methods, for instance, for mechanical tissue decellularization to create a bioactive scaffold in which new cells (e.g., exogenous or endogenous cells) can replace the old cells; introducing porosity to a site to improve cellular retention, cellular infiltration/migration, and diffusion of nutrients and signaling molecules to promote angiogenesis, cellular proliferation, and tissue regeneration similar to cell replacement therapy. Such tissue engineering methods may be useful for treating ischemic heart disease, fibrotic liver, fibrotic bowel, and traumatic spinal cord injury (SCI). For instance, for the treatment of spinal cord injury, the devices and assemblies described herein could facilitate the removal of scarred spinal cord tissue, which acts like a barrier for neuronal reconnection, before the injection of an anti-inflammatory hydrogel loaded with lentivirus to genetically engineer the spinal cord neurons to regenerate.

The elements and features of the exemplary electrode assemblies and catheters discussed above may be rearranged, recombined, and modified, without departing from the present invention. Furthermore, numerical designators such as “first”, “second”, “third”, “fourth”, etc. are merely descriptive and do not indicate a relative order, location, or identity of elements or features described by the designators. For instance, a “first” shock wave may be immediately succeeded by a “third” shock wave, which is then succeeded by a “second” shock wave. As another example, a “third” emitter may be used to generate a “first” shock wave and vice versa. Accordingly, numerical designators of various elements and features are not intended to limit the disclosure and may be modified and interchanged without departing from the subject invention.

It should be noted that the elements and features of the example catheters illustrated throughout this specification and drawings may be rearranged, recombined, and modified without departing from the present invention. For instance, while this specification and drawings describe and illustrate catheters having several example enclosure designs, the present disclosure is intended to include catheters having a variety of enclosure configurations. The number, placement, and spacing of the electrode pairs of the shock wave generators can be modified without departing from the subject invention. Further, the number, placement, and spacing of enclosures of catheters can be modified without departing from the subject invention.

It should be understood that the foregoing is only illustrative of the principles of the invention, and that various modifications, alterations, and combinations can be made by those skilled in the art without departing from the scope and spirit of the invention. Any of the variations of the various catheters disclosed herein can include features described by any other catheters or combination of catheters herein. Furthermore, any of the methods can be used with any of the catheters disclosed. Accordingly, it is not intended that the invention be limited, except as by the appended claims.

1. A method for treating a central nervous system (CNS) disease via a nasal cavity, comprising:
advancing a distal portion of a catheter through an intranasal passage to the nasal cavity such that an enclosure of the catheter is positioned at least partially within the nasal cavity, the distal portion of the catheter comprising at least one shock wave emitter that is surrounded by the enclosure, at least a portion of a surface of the enclosure coated with a drug coating, the drug coating comprising an active agent configured to treat the CNS disease;
filling the enclosure to expand the enclosure within the nasal cavity; and
with at least a portion of the enclosure spaced from tissue of the nasal cavity, generating at least one shock wave by the at least one shock wave emitter, the at least one shock wave causing a therapeutically effective amount of the active agent of the drug coating to be released from the surface of the enclosure and to be driven into the tissue of the nasal cavity such that the active agent is delivered to the CNS via the tissue of the nasal cavity and treats the CNS disease.

2. The method of claim 1, comprising generating a series of shock waves in accordance with a frequency between 1 Hz and 5 Hz.

3. The method of claim 1, wherein generating the at least one shock wave comprises generating at least one gas bubble within the enclosure that impacts an inner surface of the enclosure to eject at least a portion of the drug coating from the surface of the enclosure.

4. The method of claim 1, comprising delivering one or more voltage pulses to the at least one shock wave emitter to generate the at least one shock wave, wherein the one or more voltage pulses comprises a voltage between 1 kV and 10 kV.

5. The method of claim 1, comprising delivering one or more laser pulses to the at least one shock wave emitter to generate the at least one shock wave.

6. The method of claim 1, wherein filling the enclosure comprises pressurizing the enclosure to a pressure less than 10 atm.

7. The method of claim 1, comprising, prior to generating the at least one shock wave, delivering an irrigation solution to at least one of the intranasal passage and the nasal cavity by the catheter to clear the at least one of the intranasal passage and the nasal cavity, wherein the irrigation solution comprises saline and/or a drug.

8. The method of claim 1, wherein the intranasal passage comprises a frontal sinus ostium and the nasal cavity comprises a frontal sinus.

9. The method of claim 1, wherein generating the at least one shock wave causes the delivery of the active agent of the drug coating to the CNS via olfactory nerves.

10. The method of claim 1, wherein advancing the distal portion of the catheter through the intranasal passage comprises advancing the catheter over a guidewire.

11. (canceled)

12. (canceled)

13. The method of claim 1, wherein the drug coating comprises a crystalline form, an amorphous form, or a combination thereof.

14. The method of claim 1, wherein the drug coating comprises a plurality of micro-encapsulations containing the active agent, and wherein the plurality of micro-encapsulations comprising a plurality of microspheres and/or a plurality of microcapsules comprises a diameter between 0.5 microns and 500 microns.

15. A device for intranasal drug delivery, comprising:
an elongated tube;
at least one shock wave emitter configured to generate at least one shock wave; and
an enclosure sealed to a distal portion of the elongated tube and surrounding the at least one shock wave emitter, the enclosure fillable with a conductive fluid, and at least a portion of a surface of the enclosure coated with a drug coating, the drug coating comprising an active agent configured to treat a central nervous system (CNS) disease,
wherein the at least one shock wave emitter is configured to generate the at least one shock wave such that the at least one shock wave causes a therapeutically effective amount of the active agent of the drug coating to be released from the surface of the enclosure while the surface of the enclosure is spaced from tissue of the nasal cavity and to be driven into the tissue of the nasal cavity such that the active agent is delivered to the CNS via the tissue of the nasal cavity to treat the CNS disease.

16. The device of claim 15, wherein the at least one shock wave emitter is configured to generate the at least one shock wave configured to cause the delivery of the active agent of the drug coating to the CNS via olfactory nerves.

17. The device of claim 15, wherein the at least one shock wave emitter is configured to generate at least one gas bubble within the enclosure that impacts an inner surface of the enclosure to eject at least a portion of the drug coating from the surface of the enclosure.

18. The device of claim 15, wherein the at least one shock wave emitter is configured to generate a series of shock waves in accordance with a frequency between 1 Hz and 5 Hz.

19. The device of claim 15, wherein the enclosure is configured to be pressurized to a pressure less than 10 atm.

20. The device of claim 15, wherein the elongated tube is configured to deliver an irrigation solution to at least one of an intranasal passage and the nasal cavity to clear the at least one of the intranasal passage and the nasal cavity.

21. The device of claim 15, comprising a guidewire lumen disposed within the elongated tube and configured to receive a guidewire to guide the enclosure through an intranasal passage and to the nasal cavity.

22. The device of claim 15, wherein the elongated tube comprises a guidewire lumen configured to receive a guidewire and an irrigation lumen configured to deliver an irrigation solution to at least one of an intranasal passage and the nasal cavity, wherein the guidewire lumen is disposed within the irrigation lumen in the elongated tube, and wherein the irrigation lumen extends through a length of the enclosure to a fluid outlet at a distal end of the enclosure.

23. The device of claim 15, wherein the elongated tube is configured to couple to a syringe to fill the enclosure with the conductive fluid.

24. The device of claim 15, wherein the enclosure is fillable with an x-ray contrast agent to facilitate viewing of the enclosure.

25. The device of claim 15, wherein the enclosure comprises at least one of an elliptical balloon, spherical balloon, and a hemispherical balloon.

26. The device of claim 15, wherein at least 20% of the surface of the enclosure is coated with the drug coating.

27. The device of claim 15, wherein all of the surface of the enclosure is coated with the drug coating.

28. The device of claim 15, wherein the drug coating comprises a crystalline form, an amorphous form, or a combination thereof.

29. The device of claim 15, wherein the drug coating comprises a plurality of micro-encapsulations containing the active agent, and wherein the plurality of micro-encapsulations comprises a plurality of microspheres and/or a plurality of microcapsules comprising a diameter between 0.5 microns and 500 microns.

30. A system for intranasal drug delivery, comprising:
the device of claim 15; and
a pulse generator coupled to the at least one shock wave emitter and configured to generate energy pulses to cause the at least one shock wave emitter to generate the at least one shock wave.

31. The method of claim 1, comprising imaging the nasal cavity via an imaging sensor disposed at a distal portion of the catheter.

32. The device of claim 15, comprising an imaging sensor disposed at a distal portion of the elongated tube.
